(19) United States
(12) Patent Application Publication
(10) Pub. No.: US 2020/0316097 A1
(43) Pub. Date: Oct. 8, 2020

(54) STORAGE-STABLE READY-TO-USE INJECTABLE FORMULATIONS OF FOSAPREPITANT DIMEGLUMINE

(71) Applicants: Ravishanker Kovi, Monroe Township, NJ (US); George Roby Thomas, Winnipeg (CA); Jayaraman Kannapan, Gujarat (IN); Nirmal Chaitanya Indravadanbhai, Gujarat (IN); Patel Mitesh Manubhai, Gujarat (IN); Thupalli Ajeykumar Reddy, Bangalore (IN); Vamshi Yekkanti, Telangana (IN)

(72) Inventors: Ravishanker Kovi, Monroe Township, NJ (US); George Roby Thomas, Winnipeg (CA); Jayaraman Kannapan, Gujarat (IN); Nirmal Chaitanya Indravadanbhai, Gujarat (IN); Patel Mitesh Manubhai, Gujarat (IN); Thupalli Ajeykumar Reddy, Bangalore (IN); Vamshi Yekkanti, Telangana (IN)

(21) Appl. No.: 16/804,177

(22) Filed: Feb. 28, 2020

Related U.S. Application Data
(60) Provisional application No. 62/811,767, filed on Feb. 28, 2019.

(51) Int. Cl. A61K 31/675; A61K 47/10

(52) U.S. Cl. CPC A61K 31/675; A61K 47/10

(57) ABSTRACT

The present application provides a stable, ready-to-use fosaprepitant dimeglumine formulation which is easy to administer without need of any reconstitution step and has a desirable solubility, stability and safety profile. The concentration of the fosaprepitant dimeglumine in the liquid formulation is preferably less than about 80 mg/ml, or more preferably between about 20 mg/ml to about 60 mg/ml. In certain embodiments, the liquid formulation retains at least about 90% chemical stability of the fosaprepitant dimeglumine after storage for a commercially reasonable amount of time at a temperature between about 0° C. to about 40° C.

### FIELD OF THE INVENTION

The present application relates to a stable, ready to use, injectable fosaprepitant dimeglumine formulation.

### BACKGROUND OF THE INVENTION

Fosaprepitant for injection, commercially available under the trade name EMEND®, is a sterile, lyophilized formulation containing fosaprepitant dimeglumine, an antiemetic agent. Fosaprepitant dimeglumine has the chemical name 1-deoxy-1-(methylamino)-D-glucitol[3-[[(2R,3S)-2-[(1R)-1-[3,5bis(trifluoromethyl)phenyl]ethoxy]-3-(4-fluorophenyl)-4-morpholinyl]methyl]-2,5-dihydro-5-oxo-1H-1,2,4triazol-1-yl]phosphonate (2:1) (salt) with the empirical formula C₂₃H₂₂F₇N₄O₆P.2(C₇H₁₇NO₅). The structural formula of fosaprepitant dimeglumine is:

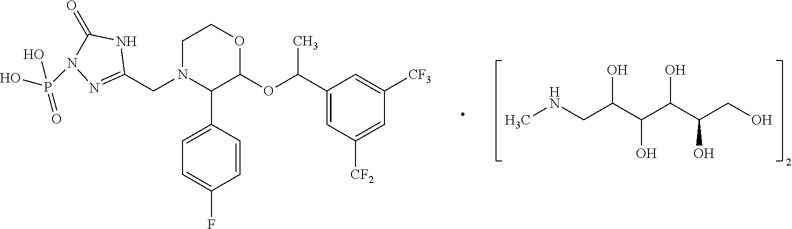

Fosaprepitant dimeglumine is a white to off-white amorphous powder with a molecular weight of 1004.83 and is freely soluble in water.

Currently, fosaprepitant dimeglumine is formulated for pharmaceutical use as a sterile dry powder filled in vials. For example, each vial of EMEND® fosaprepitant dimeglumine for injection for administration as an intravenous infusion contains 150 mg of fosaprepitant (equivalent to 245.3 mg of fosaprepitant dimeglumine) as a lyophilized powder for reconstitution and the inactive ingredients edetate disodium (5.4 mg), polysorbate 80 (75 mg), lactose anhydrous (375 mg), sodium hydroxide and/or hydrochloric acid (for pH adjustment). Fosaprepitant dimeglumine easily degrades to aprepitant unless stored at low temperature. Therefore it is conventionally supplied as a lyophilized formulation to reduce the formation of impurities and to improve the stability of the final formulation. The dry powder must be kept refrigerated at 2°−8° C., then warmed, reconstituted and diluted before it can be administered. After reconstitution and dilution, the drug is only stable for up to 24 hours at 25° C.

The currently available dosage form of fosaprepitant dimeglumine for injection is therefore costly to manufacture, distribute and store and inconvenient to use because it is not in a ready-to-use format. Therefore, an aqueous and ready-to-use fosaprepitant dimeglumine solution formulation is highly desirable, reducing manufacturing costs by eliminating the need for lyophilisation and reducing pharmacy time, labor and equipment costs by eliminating the need to reconstitute the dry powder as well as the need for refrigeration.

### SUMMARY OF THE INVENTION

Embodiments in accordance with the present disclosure provide a stable, ready-to-use injectable fosaprepitant dimeglumine solution which is easy to administer without need of any reconstitution step and has a desirable solubility, stability and safety profile.

In one or more embodiments there is provided a ready-to-use liquid parenteral formulation of fosaprepitant dimeglumine.

In one or more further embodiments, there is provided a storage-stable, ready-to-use, injectable liquid parenteral composition including fosaprepitant dimeglumine and one or more pharmaceutically acceptable solvents, co-solvents, and/or solubilizing agents.

In still further embodiments provided are ready-to-use liquid parenteral formulations including fosaprepitant dimeglumine, one or more pharmaceutically acceptable solvents, co-solvents, and/or solubilizing agents and at least one pharmaceutically acceptable excipient or adjuvant.

The storage-stable, ready-to-use, injectable compositions of the present invention may be useful as antiemetics.

In at least one aspect of the invention or inventions, a storage-stable liquid formulation is provided that includes fosaprepitant dimeglumine and a pharmaceutically acceptable vehicle.

In at least one embodiment, a concentration of the fosaprepitant dimeglumine in the liquid formulation is less than about 80 mg/ml.

In at least one embodiment, a concentration of the fosaprepitant dimeglumine in the liquid formulation is between about 20 mg/ml to about 60 mg/ml.

In at least one embodiment, the liquid formulation retains at least about 90% chemical stability of the fosaprepitant dimeglumine after storage for about six months at a temperature between about 0° C. to about 40° C.

In at least one embodiment, the vehicle comprise an NaCl solution, a concentration of the fosaprepitant dimeglumine in the liquid formulation is between about 20 mg/ml to about 60 mg/ml, and a chemical stability of the fosaprepitant dimeglumine after storage of about 32 days at room temperature is greater than 95%.

In at least one embodiment, the chemical stability of the fosaprepitant dimeglumine after storage of about four months at between about 20 C and about 80 C is greater than 99%.

In at least one embodiment, a pH of the liquid formulation is between about 7 to about 11.5.

In at least one embodiment, the storage-stable liquid formulation includes propylene glycol and ethanol.

In at least one embodiment, the propylene glycol and ethanol are present in the liquid formulation at a ratio of about 2.5:2.0.

In at least one embodiment, the propylene glycol and ethanol are present in the liquid formulation at a ratio of about 2.5:1.5.

In at least one embodiment, the propylene glycol and ethanol are present in the liquid formulation at a ratio between about 2.5:2.0 and about 2.5:1.5.

In at least one embodiment, the NaCl solution has a concentration of about 0.9%.

In at least one embodiment, the vehicle comprise an NaCl solution, a concentration of the fosaprepitant dimeglumine in the liquid formulation is between about 20 mg/ml to about 60 mg/ml, and a stability of the fosaprepitant dimeglumine after storage of about 29 days at room temperature is at least 96%.

In at least one embodiment, the storage-stable liquid formulation includes propylene glycol and wherein the propylene glycol and NaCl solution are present at a ratio of about 1:1.

In at least one embodiment, the storage-stable liquid formulation includes ethanol and wherein the ethanol and NaCl solution are present at a ratio of about 1:1.

In at least one embodiment, the stability of the fosaprepitant dimeglumine after storage of about 43 days at room temperature is at least 96%.

In at least one embodiment, the fosaprepitant dimeglumine is at least one of a pharmaceutically acceptable salt, solvate, hydrate, or a n anhydrous form thereof.

In at least one embodiment, the liquid formulation is one of a solution, suspension, or an emulsion.

The details of one or more embodiments of the invention are set forth in the description below. Other features, objects and advantages of the invention will be apparent from the description.

### DETAILED DESCRIPTION OF THE PREFERRED EMBODIMENTS

Embodiments of the present invention or inventions will now be described more fully hereinafter with reference to the accompanying examples and experiments, in which illustrative embodiments of the invention are shown. This invention(s) may, however, be embodied in many different forms and should not be construed as limited to the embodiments set forth herein; rather, these embodiments are provided so that this disclosure will be thorough and complete, and will fully convey the scope of the invention to those skilled in the art.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, “fosaprepitant dimeglumine” refers to fosaprepitant dimeglumine and the pharmaceutically acceptable salts, solvates, hydrates and anhydrous forms thereof.

As used here in “ready-to-use” when used in connection with a fosaprepitant dimeglumine formulation refers to a liquid formulation that includes fosaprepitant dimeglumine in dissolved or solubilized form and/or is intended to be used as such or upon further dilution in intravenous diluents.

As used herein, and unless otherwise specified, the term “storage-stable” refers to any liquid fosaprepitant dimeglumine-containing composition or formulation having sufficient physical and chemical stability to allow storage at a convenient temperature above the freezing point of the composition or formulation for a commercially reasonable period of time. The phrase “physical stability” refers to maintenance of colour or colourless state, dissolved oxygen level, head space oxygen level and particulate matter and the phrase “chemical stability” relates to formation of drug-related impurities in terms of total impurities, single maximum individual impurity, or maximum individual unknown impurity. For pharmaceutical products, stability is required for commercially relevant times after manufacturing, such as for about 6, 12, 18, 24, or 36 months, during which time a product is kept in its original packaging under specified storage conditions.

As used herein, and unless otherwise specified, the term “about” means an acceptable error for a particular value as determined by one of ordinary skill in the art, which depends in part on how the value is measured or determined. In certain embodiments, the term about means within 10%, 9%, 8%, 7%, 6%, 5%, 4%, 3%, 2%, 1%, 0.5%, 0.1%, or 0.05% of a given value or range.

In one or more embodiments, ready-to-use liquid parenteral formulations of fosaprepitant dimeglumine include fosaprepitant dimeglumine and one or more pharmaceutically acceptable solvents, co-solvents, and/or solubilizing agents. In other embodiments, ready-to-use liquid parenteral formulations of fosaprepitant dimeglumine include fosaprepitant dimeglumine, one or more pharmaceutically acceptable solvents, co-solvents, and/or solubilizing agents, and optionally, one or more pharmaceutically acceptable excipients or adjuvants.

Suitable pharmaceutically acceptable solvents include but are not limited to dimethylacetamide (DMA), dimethyl sulfoxide (DMSO), N-methylpyrolidone, dimethylisosorbide, ethanol, water, propylene glycol, glycerine, polyethylene alcohol, propylene glycol esters, polyethylene glycols and the like. In certain embodiments the solvent is one or more of ethanol, water, propylene glycol, glycerine and/or polyethylene glycol.

Suitable pharmaceutically acceptable co-solvents include but are not limited to ethanol, polyethylene glycol, glycerine, glycofurol and polyethylene glycol.

Suitable pharmaceutically acceptable solubilizing agents include but are not limited to cyclodextrin derivatives, alpha-cyclodextrin, beta-cyclodextrin, for example, hydroxypropyl beta cyclodextrin (HPBCD), sulfobutylether-betacyclodextrin, randomly methylated beta-cyclodextrin and the like, gamma-cyclodextrin, modified alpha-cyclodextrin, modified beta cyclodextrin, modified gamma cyclodextrin or any combination thereof.

Pharmaceutically acceptable excipients or adjuvants include but are not limited to one or more preservatives, polymers, pH adjusting agents, surfactants, chelating agents, dispersing agents, binding agents, tonicity modifying agents and antioxidants.

Examples of pharmaceutically acceptable preservatives include but are not limited to chlorobutanol, benzalkonium chloride, methyl paraben, propyl paraben, benzoic acid, sodium benzoate, sorbic acid, benzethonium chloride, cetyl pyridinium chloride, benzyl bromide, benzyl alcohol, phenylmercury nitrate, phenylmercury acetate, thiomersal, merthiolate, chlorhexidine, phenylethyl alcohol, quaternary ammonium chloride, sodium benzoate, sodium propionate, etc. and combinations thereof.

Examples of pharmaceutically acceptable polymers include but are not limited to polypropylene, polystyrene, polyvinyl chloride, polycarbonate carbomer, polycarbophil, gellan gum, cellulose derivatives, acrylates, etc. and combinations thereof.

Examples of pharmaceutically acceptable pH adjusting agents include but are not limited to sodium hydroxide, hydrochloric acid, boric acid, citric acid, acetic acid, phosphoric acid, succinic acid, potassium hydroxide, ammonium hydroxide, magnesium oxide, calcium carbonate, magnesium carbonate, magnesium aluminum silicates, malic acid, potassium citrate, sodium phosphate, lactic acid, gluconic acid, tartaric acid, fumaric acid, diethanolamine, monoethanolamine, sodium carbonate, sodium bicarbonate, triethanolamine, etc. and combinations thereof.

Examples of pharmaceutically acceptable tonicity adjusting agents include but are not limited to sodium chloride, potassium chloride, calcium chloride and magnesium chloride, glucose, glycerol, sodium hydroxide etc. and combinations thereof.

Examples of pharmaceutically acceptable surfactants include but are not limited to amphoteric, non-ionic, cationic and anionic molecules. For example, suitable surfactants include but are not limited to polysorbates, sodium lauryl sulfate, lauryl dimethyl amine oxide, docusate sodium, cetyl trimethyl ammonium bromide (CTAB), polyethoxylated alcohols, polyoxyethylene sorbitan, octoxynol, polyoxyl lauryl ether, Brij® surfactants (polyoxyethylene vegetable-based fatty ethers derived from lauryl, cetyl, stearyl and oleyl alcohols), bile salts (such as sodium deoxycholate and sodium cholate), polyoxyl castor oil, nonylphenol ethoxylate, lecithin, polyoxyethylene surfactants, polyethylene glycol esters, glycol esters of fatty acids, monoalkanolamine condensates, polyoxyethylene fatty acid amides, quaternary ammonium salts, polyoxyethylene alkyl and alicyclic amines, polyoxyethylene, sorbitan monolaurate, sorbitan stearate, Cremophor® (polyethoxylated castor oil), Solutol® (ethylene oxide/12-hydroxy stearic acid), tyloxapol, etc. and combinations thereof.

Pharmaceutically acceptable chelating agents include but are not limited to citric acid and derivatives thereof, for example, anhydrous citric acid and the like, ethylenediaminetetraacetic acid (EDTA), disodium EDTA or derivatives thereof, niacinamide or derivatives thereof, sodium deoxycholate or derivatives thereof, pentetic acid or derivatives thereof, calcium EDTA, dimercaprol, deferiprone, dimercaptosuccinic acid, etc. and combinations thereof.

Pharmaceutically acceptable vehicles include but are not limited to 0.9% Nacl, Sterile water for Injection, Dextrose, lactated ringer solution and combinations thereof.

Examples of pharmaceutically acceptable anti-oxidants include but are not limited to butylated hydroxytoluene (BHT), butylated hydroxyanisole (BHA), propyl gallate (PG), monothioglycerol, ascorbic acid, sodium ascorbate, erythorbic acid, potassium metabisulfite, sodium metabisulfite, propionic acid, sodium formaldehyde sulphoxylate, reduced glutathione, thiourea, cysteine, n-aceticysteine, methionine, sodium sulfite, alkyl gallate, vitamin E or other tocopherol analogs such as tocopherol acetate and TPGS, selenium, polyphenols, vitamin A, vitamin C etc. and combinations thereof.

The formulations according to the present invention may be in the form of clear injectable solution, suspension or emulsion.

In some embodiments the storage-stable ready-to-use injectable formulation may have a concentration of fosaprepitant of less than 80 mg/ml. In other embodiments the injectable formulation may have a concentration of fosaprepitant of from about 1 mg/ml to about 79 mg/ml. In another embodiment the injectable formulation may have a concentration of fosaprepitant of from about 20 mg/ml to about 60 mg/ml. In other embodiments the injectable formulation may have a concentration of fosaprepitant of about 50 mg/ml.

The storage-stable, ready-to-use injectable fosaprepitant dimeglumine-containing formulations disclosed herein do not require any additional reconstitution step at the time of administration.

Formulations in accordance with the present disclosure have a controlled impurity profile suitable for regulatory approval at various storage conditions. The storage-stable ready-to-use fosaprepitant dimeglumine formulations may be stored at about 0° C. to about 40° C. The storage-stable, ready-to-use fosaprepitant dimeglumine formulations for injection may retain at least 90% of the potency (e.g., chemical stability) of fosaprepitant dimeglumine after storage for six months at about 0° C. to about 40° C. temperature and 60% relative humidity.

The storage-stable, ready-to-use, injectable formulations may be formulated to provide single or multiple dosage administration. The single dosage formulation may be packaged in an ampoule, a vial, or a syringe. Multiple dosage formulations may be packaged in a vial. Multiple dosage formulations may preferably include at least one preservative.

The formulations have a pH value from about 4 to about 12. In some embodiments the pH range is from about 7 to about 12. In still other embodiments the pH range is from about 9 to about 10. In further embodiments the pH is about 8.5.

Storage-stable ready-to-use, injectable formulations disclosed herein contain fosaprepitant dimeglumine having a purity of from about 80% to about 120%. In some embodiments the formulation contains fosaprepitant dimeglumine having a purity of from about 90% to about 110%. In some embodiments the formulation contains fosaprepitant dimeglumine having a purity of about 100%.

Fosaprepitant dimeglumine is an intravenously administered antiemetic drug. It is a prodrug of aprepitant. It aids in the prevention of acute and delayed nausea and vomiting associated with chemotherapy treatment. Methods of treatment using such antiemetic drugs include administering to an individual in need thereof a therapeutically effective amount of a storage stable, ready-to-use, injectable formulation as disclosed herein.

### EXAMPLES

The following examples are for the illustration only and are not intended in any way to limit the scope of the present invention.

### Example 1

TABLE 1

| Ingredients | Qty/5 ml | |
|---|---|---|
| Fosaprepitant dimeglumine | 245.3 | mg |
| Propylene glycol | 2.5 | ml |
| Ethanol | 2.0 | ml |
| Trisodium Orthophosphate Buffer | 0.5 | ml |

The ingredients in Table 1 were employed as follows: Fosaprepitant dimeglumine was added to a manufacturing vessel containing 0.9% NaCl solution and the mixture stirred to obtain a clear solution. To the above obtained clear solution ethanol and propylene glycol were added and the mixture was stirred to get a uniformly distributed solution. The pH of the solution was found to be in between 7-9.5. The obtained solution was filtered and filled in vials, followed by capping and sealing of the vials. The formulation was tested for stability at room temperature for a period of 32 days and at 2-8° C. condition for 4 months. Stability data is summarized in Table 1A.

TABLE 1A

| | | RT | 2-8° C. |
|---|---|---|---|
| Stability at Day 32 | RRT | (day 32) | (4 months) |
| Purity | 1.00 | 95.51 | 99.32 |
| Fosaprepitant Desfluoro Impurity | 0.88 | 0.07 | 0.06 |
| Aprepitant | 2.00 | 4.32 | 0.57 |
| Fosaprepitant Benzyl Ester | 1.79 | 0.06 | 0.03 |
| Maximum Individual impurity | 0.76 | 0.02 | 0.02 |
| Total Impurities | | 4.49 | 0.68 |

### Example 2

TABLE 2

| Ingredients | Qty/5 ml | |
|---|---|---|
| Fosaprepitant dimeglumine | 245.3 | mg |
| Propylene glycol | 2.5 | ml |
| Ethanol | 1.5 | ml |
| Trisodium Orthophosphate Buffer | 1.0 | ml |

The ingredients in Table 2 were employed as follows: Fosaprepitant dimeglumine was added to a manufacturing vessel containing 0.9% NaCl solution and the mixture stirred to obtain a clear solution. To the above obtained clear solution ethanol and propylene glycol were added and the mixture stirred to get a uniformly distributed solution. The pH of the solution was found to be in between 8-11.5. The obtained solution was filtered and filled in vials, followed by capping and sealing of the vials. The formulation was tested for stability at room temperature for a period of 32 days and at 2-8° C. for 4 months. Stability data is summarized in Table 2A

TABLE 2A

| | | RT | 2-8° C. |
|---|---|---|---|
| Stability at Day 32 | RRT | (day 32) | (4 months) |
| Purity | 1.00 | 96.03 | 99.55 |
| Fosaprepitant Desfluoro Impurity | 0.88 | 0.08 | 0.05 |
| Aprepitant | 2.00 | 3.81 | 0.37 |
| Fosaprepitant Benzyl Ester | 1.79 | 0.06 | 0.02 |
| Maximum Individual impurity | 0.76 | 0.02 | 0.01 |
| Total Impurities | | 3.97 | 0.45 |

### Example 3

TABLE 3

| Ingredients | Qty/5 ml | |
|---|---|---|
| Fosaprepitant dimeglumine | 245.3 | mg |
| Propylene glycol | 2.5 | ml |
| 0.9% NaCl solution | 2.5 | ml |

The ingredients in Table 3 were employed as follows: Fosaprepitant dimeglumine was added to a manufacturing vessel containing 0.9% NaCl solution and the mixture stirred to obtain a clear solution. To the above obtained clear solution propylene glycol was added and the mixture stirred to get a uniformly distributed solution. The pH of the solution was found to be in between 4.5-8.5. The obtained solution was filtered and filled in vials, followed by capping and sealing of the vials. The formulation was tested for stability at room temperature for a period of 29 days and at 2-8° C. for 4 months. Stability data is summarized in Table 3A

TABLE 3A

| | | RT | 2-8° C. |
|---|---|---|---|
| Stability at Day 29 day | RRT | (day 29) | (4 months) |
| Purity | 1.00 | 97.00 | 99.48 |
| Fosaprepitant Desfluoro Impurity | 0.88 | 0.08 | 0.06 |
| Aprepitant | 2.00 | 2.81 | 0.41 |
| Fosaprepitant Benzyl Ester | 1.79 | 0.05 | 0.03 |
| Maximum Individual impurity | 1.84 | 0.03 | 0.02 |
| Total Impurities | | 3.00 | 0.52 |

### Example 4

TABLE 4

| Ingredients | Qty/5 ml | |
|---|---|---|
| Fosaprepitant dimeglumine | 245.3 | mg |
| Ethanol | 2.5 | ml |
| 0.9% NaCl solution | 2.5 | ml |

The ingredients in Table 4 were employed as follows: Fosaprepitant dimeglumine was added to a manufacturing vessel containing 0.9% NaCl solution and the mixture stirred to obtain a clear solution. To the above obtained clear solution ethanol was added and the mixture stirred to get a uniformly distributed solution. The pH of the solution was found to be in between 4.5-8.5. The obtained solution was filtered and filled in vials, followed by capping and sealing of the vials. The formulation was tested for stability at room temperature for a period of 43 days. Stability data is summarized in Table 4A

TABLE 4A

| Stability at Day 43 day | RRT | RT (day 43) |
|---|---|---|
| Purity | 1.00 | 96.03 |
| Fosaprepitant Desfluoro Impurity | 0.88 | 0.08 |
| Aprepitant | 2.00 | 3.80 |
| Fosaprepitant Benzyl Ester | 1.79 | 0.06 |
| Maximum Individual impurity | 0.74 | 0.02 |
| Total Impurities | | 3.97 |

### Example 5

TABLE 5

| Ingredients | Qty/5 ml | |
|---|---|---|
| Fosaprepitant dimeglumine | 245.3 | mg |
| Propylene glycol | 2.5 | ml |
| 0.9% NaCl solution | 2.2 | ml |
| 0.1N NaOH | 0.3 | ml |

The ingredients in Table 5 were employed as follows: Fosaprepitant dimeglumine was added to a manufacturing vessel containing 0.9% NaCl solution and the mixture stirred to obtain a clear solution. To the above obtained clear solution propylene glycol was added and the mixture stirred to get a uniformly distributed solution, 0.1N NaOH was added for pH adjustment. The pH of the solution after adjustment was found to be between 7.0-11.5. The obtained solution was filtered and filled in vials, followed by capping and sealing of the vials. The formulation was tested for stability at 40° C. for a period of 5 days. Stability data is summarized in Table 5A

TABLE 5A

| Stability at Day 5 | RRT | 40° C. (day 5) |
|---|---|---|
| Purity | 1.00 | 97.58 |
| Fosaprepitant Desfluoro Impurity | 0.88 | 0.08 |
| Aprepitant | 2.00 | 2.27 |
| Fosaprepitant Benzyl Ester | 1.79 | 0.05 |
| Maximum Individual impurity | 0.74 | 0.02 |
| Total Impurities | | 2.42 |

### Example 6

TABLE 6

| Ingredients | Qty/5 ml | |
|---|---|---|
| Fosaprepitant dimeglumine | 245.3 | mg |
| Ethanol | 2.5 | ml |
| Water | 2.3 | ml |
| 0.1N NaOH | 0.2 | ml |

The ingredients in Table 6 were employed as follows: Fosaprepitant dimeglumine was added to a manufacturing vessel containing water and the mixture stirred to obtain a clear solution. To the above obtained clear solution ethanol was added and the mixture was stirred to get a uniformly distributed solution, 0.1N NaOH was added for pH adjustment. The pH of the solution after adjustment was found to be in between 7.0-11.0. The obtained solution was filtered and filled in vials, followed by capping and sealing of the vials. The formulation was tested for stability at 40° C. for a period of 8 days. Stability data is summarized in Table 6A

TABLE 6A

| Stability at Day 8 40° C. | RRT | 40° C. (day 8) |
|---|---|---|
| Purity | 1.00 | 97.69 |
| Fosaprepitant Desfluoro Impurity | 0.88 | 0.07 |
| Aprepitant | 2.00 | 2.16 |
| Fosaprepitant Benzyl Ester | 1.79 | 0.05 |
| Maximum Individual impurity | 0.73 | 0.02 |
| Total Impurities | | 2.31 |

### Example 7

TABLE 7

| Ingredients | Qty/5 ml | |
|---|---|---|
| Fosaprepitant dimeglumine | 245.3 | mg |
| Ethanol | 2.5 | ml |
| Water | 2.2 | ml |
| 0.1N NaOH | 0.3 | ml |

The ingredients in Table 7 were employed as follows: Fosaprepitant dimeglumine was added to a manufacturing vessel containing water and the mixture stirred to obtain a clear solution. To the above obtained clear solution ethanol was added and the mixture was stirred to get a uniformly distributed solution, 0.1N NaOH was added for pH adjustment. The pH of the solution after adjustment was found to be in between 8.0-12.0. The obtained solution was filtered and filled in vials, followed by capping and sealing of the vials. The formulation was tested for stability at 40° C. for a period of 8 days. Stability data is summarized in Table 7A

TABLE 7A

| Stability at Day 8 40° C. | RRT | 40° C. (day 8) |
|---|---|---|
| Purity | 1.00 | 97.58 |
| Fosaprepitant Desfluoro Impurity | 0.88 | 0.07 |
| Aprepitant | 2.00 | 2.27 |
| Fosaprepitant Benzyl Ester | 1.79 | 0.06 |
| Maximum Individual impurity | 0.73 | 0.02 |
| Total Impurities | | 2.42 |

### Example 8

TABLE 8

| Ingredients | Qty/5 ml | |
|---|---|---|
| Fosaprepitant dimeglumine | 245.3 | mg |
| Water | 4.9 | ml |
| 0.1N NaOH | 0.1 | ml |

The ingredients in Table 8 were employed as follows: Fosaprepitant dimeglumine was added to a manufacturing vessel containing water and the mixture stirred to obtain a clear solution. To the above obtained clear solution 0.1N NaOH was added for pH adjustment. The pH of the solution after adjustment was found to be between 7.0-11.0. The obtained solution was filtered and filled in vials, followed by capping and sealing of the vials. The formulation was tested for stability at room temperature and at 40° C. for a period of 5 days. Stability data is summarized in Table 8A

TABLE 8A

| Stability at Day 5 | RRT | RT ((day 5)) | 40° C. (day 5) |
|---|---|---|---|
| Purity | 1.00 | 99.19 | 98.38 |
| Fosaprepitant Desfluoro Impurity | 0.88 | 0.07 | 0.08 |
| Aprepitant | 2.00 | 0.60 | 1.45 |
| Fosaprepitant Benzyl Ester | 1.79 | 0.06 | 0.07 |
| Maximum Individual impurity | 0.73 | 0.02 | 0.03 |
| Total Impurities | | 0.81 | 1.62 |

### Example 9

TABLE 9

| Ingredients | Qty/5 ml | |
|---|---|---|
| Fosaprepitant dimeglumine | 245.3 | mg |
| 0.9% NaCl solution | 4.9 | ml |
| 0.1N NaOH | 0.1 | ml |

The ingredients in Table 9 were employed as follows: Fosaprepitant dimeglumine was added to a manufacturing vessel containing 0.9% NaCl solution and the mixture stirred to obtain a clear solution. To the above obtained clear solution 0.1N NaOH was added for pH adjustment. The pH of the solution after adjustment was found to be between 7.0-11.5. The obtained solution was filtered and filled in vials, followed by capping and sealing of the vials. The formulation was tested for stability at room temperature and at 40° C. for a period of 5 days. Stability data is summarized in Table 9A

TABLE 9A

| | | RT | |
|---|---|---|---|
| Stability at Day 5 | RRT | (day 5) RT | 40° C. (day 5) |
| Purity | 1.00 | 99.29 | 98.62 |
| Fosaprepitant Desfluoro Impurity | 0.88 | 0.07 | 0.08 |
| Aprepitant | 2.00 | 0.55 | 1.23 |
| Fosaprepitant Benzyl Ester | 1.79 | 0.05 | 0.05 |
| Maximum Individual impurity | 0.73 | 0.02 | 0.02 |
| Total Impurities | | 0.71 | 1.38 |

### Example 10

TABLE 10

| Ingredients | Qty/5 ml | |
|---|---|---|
| Fosaprepitant dimeglumine | 245.3 | mg |
| Water | 4.85 | ml |
| 0.1N NaOH | 1.15 | ml |

The ingredients in Table 10 were employed as follows: Fosaprepitant dimeglumine was added to a manufacturing vessel containing water and the mixture stirred to obtain a clear solution. To the above obtained clear solution 0.1N NaOH was added for pH adjustment. The pH of the solution after adjustment was found to be between 7.0-12.0. The obtained solution was filtered and filled in vials, followed by capping and sealing of the vials. The formulation was tested for stability at room temperature and at 40° C. for a period of 5 days. Stability data is summarized in Table 10A

TABLE 10A

| Stability at Day 5 | RRT | RT (day 5) | 40° C. (day 5) |
|---|---|---|---|
| Purity | 1.00 | 99.21 | 98.43 |
| Fosaprepitant Desfluoro Impurity | 0.88 | 0.08 | 0.08 |
| Aprepitant | 2.00 | 0.61 | 1.41 |
| Fosaprepitant Benzyl Ester | 1.79 | 0.06 | 0.06 |
| Maximum Individual impurity | 0.73 | 0.02 | 0.02 |
| Total Impurities | | 0.79 | 1.57 |

### Example 11

TABLE 11

| Ingredients | Qty/5 ml | |
|---|---|---|
| Fosaprepitant dimeglumine | 245.3 | mg |
| 0.9% NaCl solution | 4.85 | ml |
| 0.1N NaOH | 0.15 | ml |

The ingredients in Table 11 were employed as follows: Fosaprepitant dimeglumine was added to a manufacturing vessel containing 0.9% NaCl solution and the mixture stirred to obtain a clear solution. To the above obtained clear solution 0.1N NaOH was added for pH adjustment. The pH of the solution after adjustment was found to be between 7.0-12.0. The obtained solution was filtered and filled in vials, followed by capping and sealing of the vials. The formulation was tested for stability at room temperature and at 40° C. for a period of 5 days. Stability data is summarized in Table 11A

TABLE 11A

| Stability at Day 5 | RRT | RT (day 5) | 40° C. (day 5) |
|---|---|---|---|
| Purity | 1.00 | 99.26 | 98.60 |
| Fosaprepitant Desfluoro Impurity | 0.88 | 0.08 | 0.08 |
| Aprepitant | 2.00 | 0.58 | 1.24 |
| Fosaprepitant Benzyl Ester | 1.79 | 0.05 | 0.08 |
| Maximum Individual impurity | 0.73 | 0.03 | 0.02 |
| Total Impurities | | 0.74 | 1.40 |

### Example 12

TABLE 12

| Ingredients | Qty/5 ml | |
|---|---|---|
| Fosaprepitant dimeglumine | 245.3 | mg |
| 0.9% NaCl solution | 5.0 | ml |

Fosaprepitant dimeglumine was added to a manufacturing vessel containing 0.9% NaCl solution and the mixture stirred to obtain a clear solution. The obtained clear solution was filled in vials, followed by capping and sealing. The formulation was tested for stability at 2-8° C. for 4 months. Stability data is summarized in Table 12A

TABLE 12A

| Stability at 4 M | RRT | 2-8° C. (4 months) |
|---|---|---|
| Purity | 1.00 | 99.71 |
| Fosaprepitant Desfluoro Impurity | 0.88 | 0.06 |
| Aprepitant | 2.00 | 0.18 |
| Fosaprepitant Benzyl Ester | 1.79 | 0.03 |
| Maximum Individual impurity | 0.73 | 0.02 |
| Total Impurities | | 0.29 |

While the foregoing invention has been described in some detail for purposes of clarity and understanding, it will be appreciated by one skilled in the art, from a reading of the disclosure, that various changes in form and detail can be made without departing from the true scope of the invention.

What is claimed is:

1. A storage-stable liquid formulation comprising fosaprepitant dimeglumine and a pharmaceutically acceptable vehicle.

2. The storage-stable liquid formulation of claim 1, wherein a concentration of the fosaprepitant dimeglumine in the liquid formulation is less than about 80 mg/ml.

3. The storage-stable liquid formulation of claim 1, wherein a concentration of the fosaprepitant dimeglumine in the liquid formulation is between about 20 mg/ml to about 60 mg/ml.

4. The storage-stable liquid formulation of claim 1, wherein the liquid formulation retains at least about 90% chemical stability of the fosaprepitant dimeglumine after storage for about six months at a temperature between about 0° C. to about 40° C.

5. The storage-stable liquid formulation of claim 1, wherein the vehicle comprise an NaCl solution, a concentration of the fosaprepitant dimeglumine in the liquid formulation is between about 20 mg/ml to about 60 mg/ml, and a chemical stability of the fosaprepitant dimeglumine after storage of about 32 days at room temperature is greater than 95%.

6. The storage-stable liquid formulation of claim 5, wherein the chemical stability of the fosaprepitant dimeglumine after storage of about four months at between about 2° C. and about 8° C. is greater than 99%.

7. The storage-stable liquid formulation of claim 5, wherein a pH of the liquid formulation is between about 7 to about 11.5.

8. The storage-stable liquid formulation of claim 5, comprising propylene glycol and ethanol.

9. The storage-stable liquid formulation of claim 8, wherein the propylene glycol and ethanol are present in the liquid formulation at a ratio of about 2.5:2.0.

10. The storage-stable liquid formulation of claim 8, wherein the propylene glycol and ethanol are present in the liquid formulation at a ratio of about 2.5:1.5.

11. The storage-stable liquid formulation of claim 8, wherein the propylene glycol and ethanol are present in the liquid formulation at a ratio between about 2.5:2.0 and about 2.5:1.5.

12. The storage-stable liquid formulation of claim 5, wherein the NaCl solution has a concentration of about 0.9%.

13. The storage-stable liquid formulation of claim 1, wherein the vehicle comprise an NaCl solution, a concentration of the fosaprepitant dimeglumine in the liquid formulation is between about 20 mg/ml to about 60 mg/ml, and a stability of the fosaprepitant dimeglumine after storage of about 29 days at room temperature is at least 96%.

14. The storage-stable liquid formulation of claim 13, comprising propylene glycol and wherein the propylene glycol and NaCl solution are present at a ratio of about 1:1.

15. The storage-stable liquid formulation of claim 13, comprising ethanol and wherein the ethanol and NaCl solution are present at a ratio of about 1:1.

16. The storage-stable liquid formulation of claim 13, wherein the stability of the fosaprepitant dimeglumine after storage of about 43 days at room temperature is at least 96%.

17. The storage-stable liquid formulation of claim 1, wherein the fosaprepitant dimeglumine is at least one of a pharmaceutically acceptable salt, solvate, hydrate, or a n anhydrous form thereof.

18. The storage-stable liquid formulation of claim 1, wherein the liquid formulation is one of a solution, suspension, or an emulsion.
